(19) United States
(12) Patent Application Publication
(10) Pub. No.: US 2009/0014462 A1
(43) Pub. Date: Jan. 15, 2009

(54) NEEDLE DISPENSER SYSTEM AND HAND TOOL FOR SAME

(75) Inventors: Richard Costa, Bedminster, NJ (US); Hung Mach, Fort Lee, NJ (US)

Correspondence Address: DUANE MORRIS LLP - Philadelphia;IP DEPARTMENT, 30 SOUTH 17TH STREET, PHILADELPHIA, PA, 19103-4196, US

(73) Assignee: PHARMADESIGN, INC., Warren, NJ (US)

(21) Appl. No.: 11/775,630

(22) Filed: Jul. 10, 2007

(51) Int. Cl. A61M 5/34

(52) U.S. Cl. 221/185; 206/365

(57) ABSTRACT

A needle dispensing system including a pen-type hypodermic syringe having a threaded end that is suitable for mating with a needle assembly having a threaded portion that is complementary to the threaded end of the syringe and located within a closed pod. A hand tool is provided for controlling the pod that includes a collet sized so as to releasably grip a portion of the closed pod for opening. The hand tool also includes a radiused guard that helps to prevent inadvertent needle sticks during use, and a biased release button the actuation of which causes the opened pod to be released from the collet. A hand tool is also provided for controlling a container holding a disposable needle assembly that includes a housing that supports a collet that is sized so as to releasably grip a portion of the container for opening, and a biased release button the actuation of which causes the opened pod to be released from the collet.

### FIELD OF THE INVENTION

The present invention generally relates to patient controlled injection systems and, more particularly to patient operated hypodermic syringes that often have the general appearance of a pen and that require periodic removal and replacement of a needle portion of the hypodermic syringe.

### BACKGROUND OF THE INVENTION

Home treatment methods for the control and management of various diseases are becoming more popular, with high success rates being achieved for treatment of diabetes when a patient controls the disease by self-testing blood glucose levels and administering a correct dose of insulin. Between doctor's office visits, the patient is responsible for carrying out the prescribed regimen, which often includes frequent insulin injections using a syringe. Traditionally, patients were required to insert the needle of the syringe into a separate medication vial to withdraw medication, and once the medication is withdrawn from the vial, remove any air bubbles or extra medication, and then inject the medication.

Typical syringes were not be preloaded with medication; thus, requiring the patient to carry a separate medication vial. Also, people with dexterity disorders often had difficulty lining up the needle portion of the syringe with the rubber septum on the medication vial. This lead to unintentional needle pricks or excessive time being required to complete an injection. Pen-type injectors that often incorporate prepackaged insulin have been developed for the foregoing patients. A disadvantage to pen-type injectors is that the disposable needles must be removed from the pen injector assembly after use. Individuals with limited dexterity or eyesight have often found this procedure to be difficult, and sometimes dangerous, due to unintentional needle jabs.

### SUMMARY OF THE INVENTION

In one embodiment, the present invention provides a needle dispensing system including a pen-type hypodermic syringe having a threaded end that is suitable for mating with a needle assembly having a threaded portion that is complementary to the threaded end of the syringe and located within a closed pod. A hand tool is provided for controlling the pod that includes a collet sized so as to releasable grip a portion of the closed pod for opening. The hand tool also includes a biased release button the actuation of which causes the opened pod to be released from the collet.

In another embodiment of the invention, a hand tool is provided for controlling a container holding a disposable needle assembly that includes a housing that supports a collet that is sized so as to releasably grip a portion of the container for opening, and a biased release button the actuation of which causes the opened pod to be released from the collet.

### BRIEF DESCRIPTION OF THE DRAWINGS

These and other features and advantages of the present invention will be more fully disclosed in, or rendered obvious by, the following detailed description of the preferred embodiment of the invention, which is to be considered together with the accompanying drawings wherein like numbers refer to like parts and further wherein:

FIG. 1 is a perspective view of a needle dispenser system formed in accordance with one embodiment of the invention, with a pen-type personal syringe having a needle assembly threaded onto a distal end, and positioned above a needle-pod receptacle having a now empty needle-pod received within a receptacle opening;

FIG. 2 is a broken-away, perspective view of a needle assembly threaded onto a distal end of the pen-type personal syringe system shown in FIG. 1;

FIG. 3 is a broken-away, perspective view of the pen-type personal syringe system shown in FIG. 2, with a needle guard removed from a distal portion of a hypodermic needle;

FIG. 4 is a perspective view of a pen-type personal syringe system of the type used in connection with the present invention;

FIG. 5 is a perspective view of a pen-type personal syringe system of the type used in connection with the present invention with its cap removed;

FIG. 6 is a perspective view of a needle-pod receptacle formed in accordance with the present invention having a needle-pod positioned above it ready for insertion;

FIG. 7 is a cross-sectional view of a needle-pod and needle-pod receptacle shown in FIG. 6;

FIG. 8 is a perspective view of a needle-pod receptacle having a needle-pod received within a receptacle opening;

FIG. 9 is a perspective view similar to that shown in FIG. 8, with a foil seal being removed so as to provide access to the needle assembly within the needle-pod;

FIG. 10 is a perspective view of a needle-pod receptacle formed in accordance with the present invention;

FIG. 11 is a side view of the needle-pod receptacle shown in FIG. 10;

FIG. 12 is a further side view of the needle-pod receptacle shown in FIG. 10, rotated by about ninety degrees from the side view shown in FIG. 11;

FIG. 13 is a top view of the needle-pod receptacle shown in FIG. 10;

FIG. 14 is a bottom view of the needle-pod receptacle shown in FIG. 10;

FIG. 15 is a cross-sectional view of a needle-pod receptacle having a needle-pod received within a receptacle opening as shown in FIG. 8;

FIG. 16 is a perspective view of a pen-type personal syringe system of the type used in connection with the present invention, with its cap removed, and positioned above a needle-pod receptacle having a needle-pod received within a receptacle opening;

FIG. 17 is a perspective view of a pen-type personal syringe system of the type used in connection with the present invention being rotated into threaded engagement with a portion of a needle-pod received within a receptacle opening of a needle-pod receptacle;

FIG. 18 is a broken-away, cross-sectional view of the pen-type personal syringe system arranged in threaded engagement with a portion of a needle-pod received within a receptacle opening of a needle-pod receptacle, as shown in FIG. 17;

FIG. 19 is a perspective view of the pen-type personal syringe system, with a distal portion of a hypodermic needle exposed for use;

FIG. 20 is a perspective view of a pen-type personal syringe system, with a distal portion of a hypodermic needle exposed after use, and positioned above a needle-pod receptacle having a needle-pod received within a receptacle opening;

FIG. 21 is a broken-away, cross-sectional view of the pen-type personal syringe system arranged in threaded engagement with a portion of a needle-pod received within a receptacle opening of a needle-pod receptacle;

FIG. 22 is a perspective view of a needle-pod receptacle having a needle-pod received within a receptacle opening;

FIG. 23 is a bottom view of the needle-pod receptacle shown in FIGS. 21 and 22;

FIG. 24 a cross-sectional view of a needle-pod receptacle having a refilled needle-pod received within a receptacle opening with a finger about to actuate the pod-release assembly; and

FIG. 25 a cross-sectional view of a needle-pod receptacle similar to that shown in FIG. 24, having showing the pod-release assembly fully actuated so as to release the refilled needle-pod from within the receptacle opening of the needle-pod receptacle.

### DETAILED DESCRIPTION OF THE PREFERRED EMBODIMENT

This description of preferred embodiments is intended to be read in connection with the accompanying drawings, which are to be considered part of the entire written description of this invention. The drawing figures are not necessarily to scale and certain features of the invention may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures.

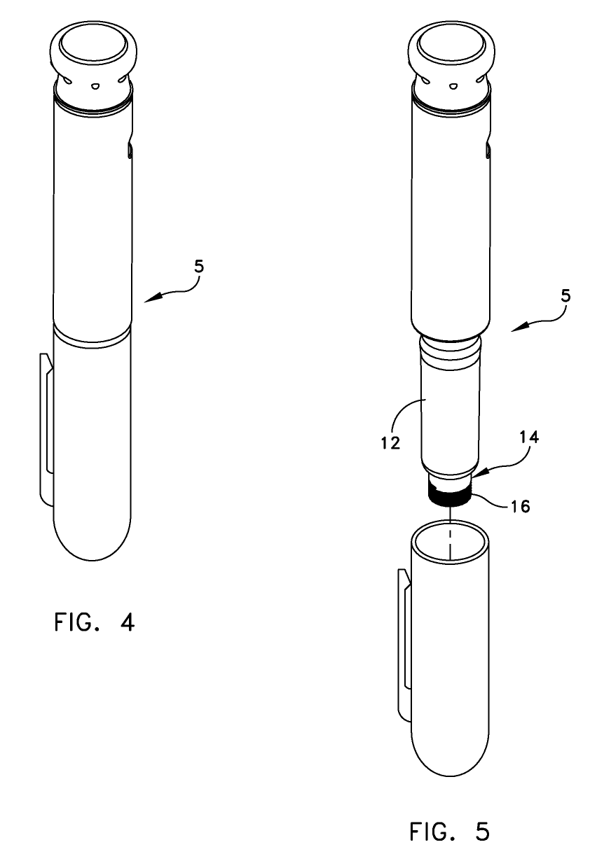

Referring to FIGS. 1-6, needle dispenser system 3 is provided that includes a patient operated hypodermic syringe 5, that often has the general appearance of a pen, and requires periodic replacement of a needle assembly 7 that is provided to the patient safely housed within a needle-pod 9, i.e., in a protective case or closed receptacle that is suitable for transporting a disposable hypodermic needle in a sterile and untampered-with condition. The periodic removal, replacement, and disposal of needle assembly 7 is facilitated through use of needle-pod-tool 10. In some embodiments of the invention, a patient operated hypodermic syringe 5 generally includes a multi-piece housing 12 that is often adapted to receive liquid 13 such as a dose of medication, e.g., insulin, antidote, etc. A distal end 14 of housing 12 includes a threaded portion 16. Exemplary patient operated hypodermic syringes of the type contemplated for use in the present invention may be found in U.S. Pat. Nos. 6,562,011; 6,221,046; 5,820,602; 5,584,815; 5,454,786; 5,334,162; 5,308,340; 5,295,976; 5,228,883; 5,226,895; 5,226,896, which patents are hereby incorporated herein by reference.

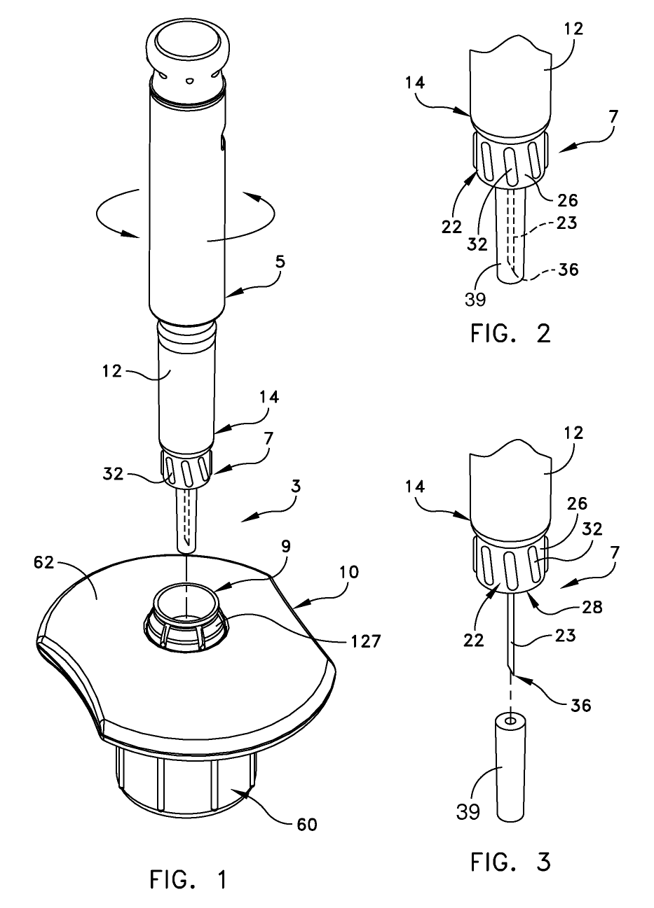

Referring initially to FIGS. 2-3 and 15, needle assembly 7 includes cylindrical header 22 and a hypodermic needle 23. Cylindrical header 22 has a blind recess 24 defined by an annular wall 26 that projects outwardly from the peripheral edge of a substantially circular base wall 28 (FIG. 15). The inner surface of annular wall 26 includes a raised thread 30, while the outer surface of annular wall 26 comprises one or more raised structures, e.g., a plurality of ribs 32. Hypodermic needle 23 comprises an elongate, substantially cylindrical steel tube, having a proximal end and a sharpened distal end 36 that permits subcutaneous or intramuscular injection. Hypodermic needle 23 is lodged within a central portion of base wall 28 so that the proximal end projects outwardly from base wall 28 within blind recess 24, and sharpened distal end 36 projects outwardly from the outer surface of base wall 28. A separable needle safety cap 39 is often positioned over sharpened distal end 36 of hypodermic needle 23.

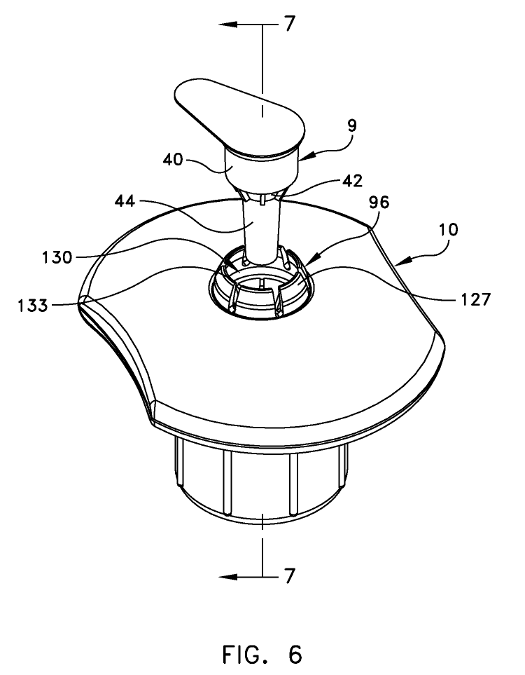

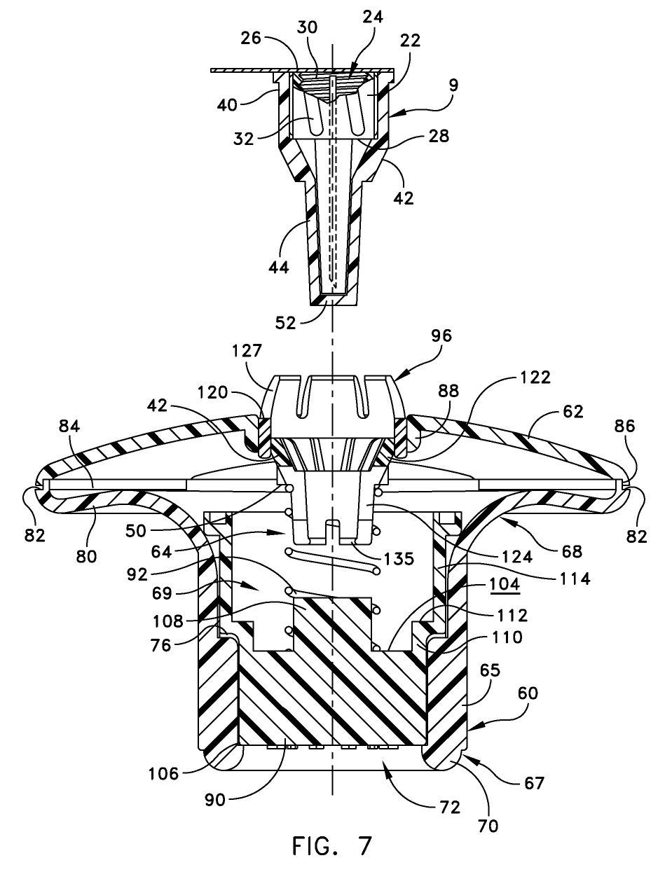

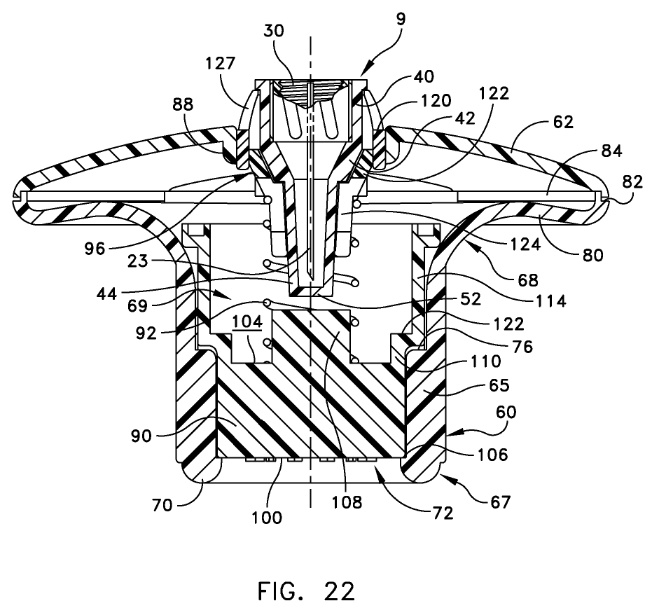

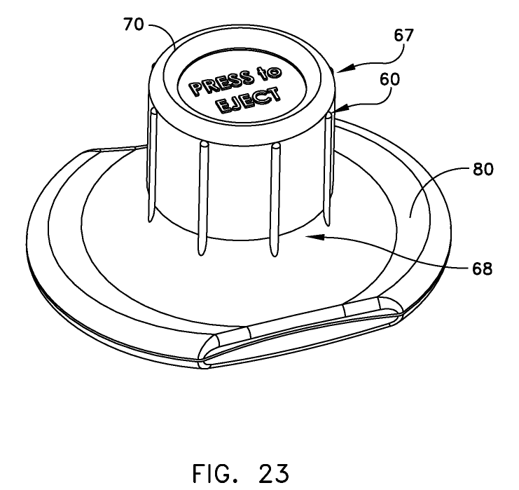

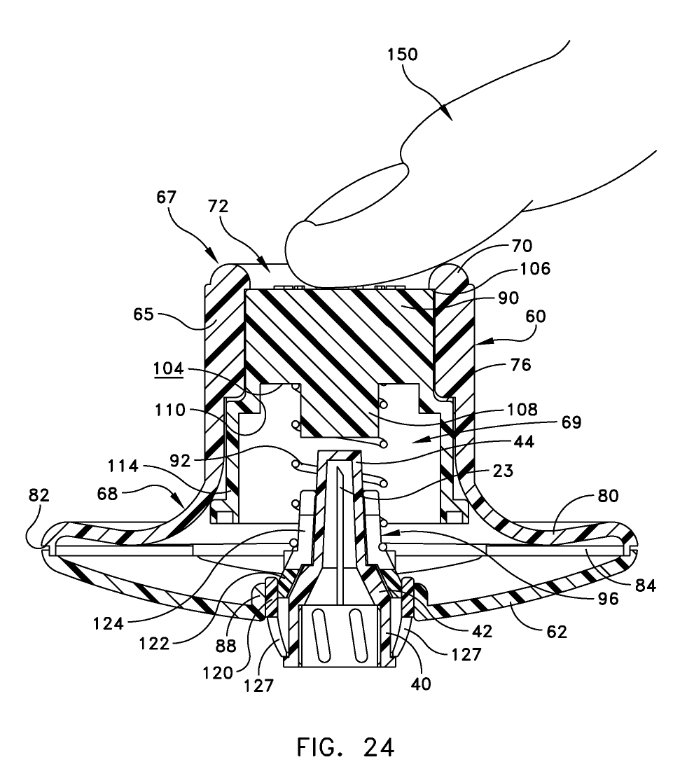

Referring to FIGS. 6-9, 15-16, 18, 21-22, and 24-25, needle assembly 7 is protectively housed within needle-pod 9 which generally comprises a blind receptacle formed by a first cylindrical wall 40, an intermediate tapered wall 42, and a second cylindrical wall 44. First cylindrical wall 40 defines an open end 45 that may be covered with a peel-away foil shield 47 that includes a tab 48 for easy removal from needle-pod 9. First cylindrical wall 40 defines an inner diameter that is somewhat larger than the diameter of annular wall 26 of needle assembly 7, but somewhat smaller than the diameter of annular wall 26 plus the one or more raised structures, such as plurality of ribs 32. In this way, annular wall 26 may be snugly press-fit within open end 45 of needle-pod 9. Intermediate wall 42 projects radially inwardly from the bottom end of first cylindrical wall 40, and defines an annular shoulder 50. Second cylindrical wall 44 projects outwardly from the center of shoulder 50, has an end wall 52, and is often somewhat longer and substantially larger in diameter that sharpened distal end 36 of hypodermic needle 23.

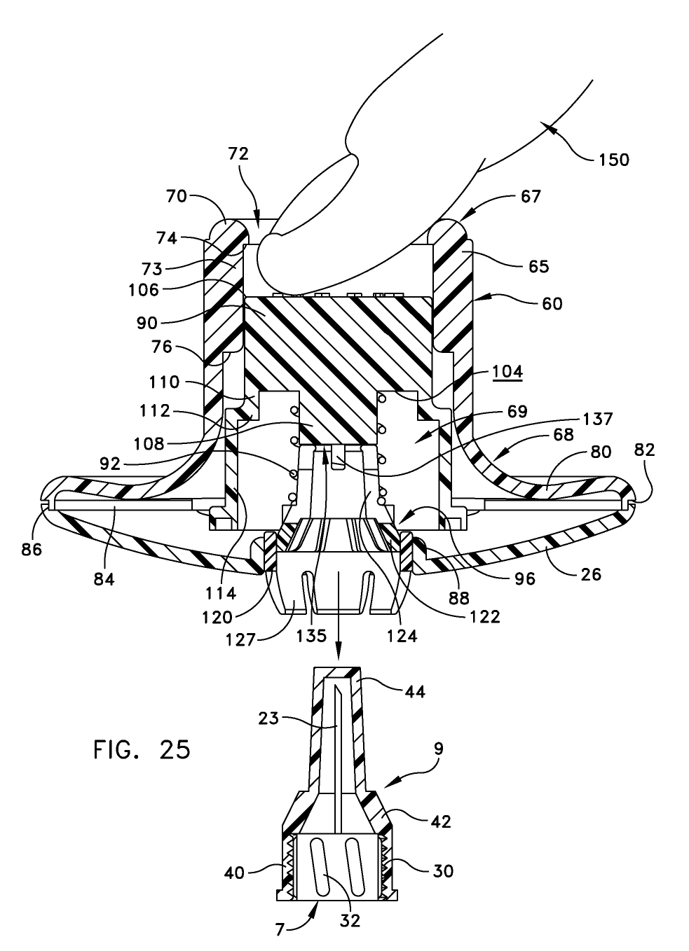

Referring to FIGS. 1, 6-9, 15, 22, 24, and 25, needle-pod-tool 10 comprises a handle 60, a face plate 62, and a pod handling assembly 64. More particularly, handle 60 includes a wall 65 having a proximal end 67, a distal end 68, and which defines an interior chamber 69. In preferred embodiments, proximal portions of wall 65 have a substantially cylindrical, ergonomic shape that is suitably sized for gripping comfortably within a patient's hand. Wall 65 has a rounded proximal end edge 70 that has a first inner diameter and defines an opening 72 that is in communication with interior chamber 69. The first inner diameter of proximal end edge 70 is somewhat smaller than the inner diameter of the next adjacent section 73 of wall 65 so as to define a first annular shoulder 74 (FIG. 25). Wall 65 has a third inner diameter at distal end 68 that is somewhat larger than the inner diameter of section 73 so as to define a second inner annular shoulder 76.

The distal-most portions of wall 65 flair radially outwardly so as to form a hand guard 80. A peripheral shoulder 82 is circumferentially defined on the peripheral edge of hand guard 80 by an annular wall 84 that projects substantially upwardly from the distal-most, flared portions of wall 65. Hand guard 80 often includes a chordal or inwardly curved edge 81 (FIGS. 12-14) and is sized so as to provide adequate protection for the user's hand during operation so as to avoid needle sticks. Face plate 62 comprises a convex disk having an outer diameter that is substantially equal to the diameter of hand guard 80. A central opening is defined through face plate 62 by an annular wall 88 that projects outwardly from the rear, concave surface of face plate 62. A peripheral recessed groove 86 is circumferentially defined in the inner peripheral edge of face plate 62 that is complementary to annular wall 84 of hand guard 80. Face plate 62 also often includes a chordal or inwardly curved edge 85.

Pod handling assembly 64 includes a plunger 90, a spring 92, and a pod-collet 96. Plunger 90 includes an ejector pad 100 having an outer surface 102 (FIG. 14) an inner surface 104. Outer surface 102 is limited in the radial direction by peripheral edge 106. A post 108 projects outwardly from inner surface 104, and is suitably sized to be received within a proximal end of a helical spring 92. An annular wall 110 projects upwardly from the peripheral edge of inner surface 104 in radially spaced relation to post 108. An annular ledge 112 projects radially outwardly from the outer surface of annular wall 110. An annular wall 114 projects upwardly from the peripheral edge of annular ledge 112 in radially spaced relation to post 108.

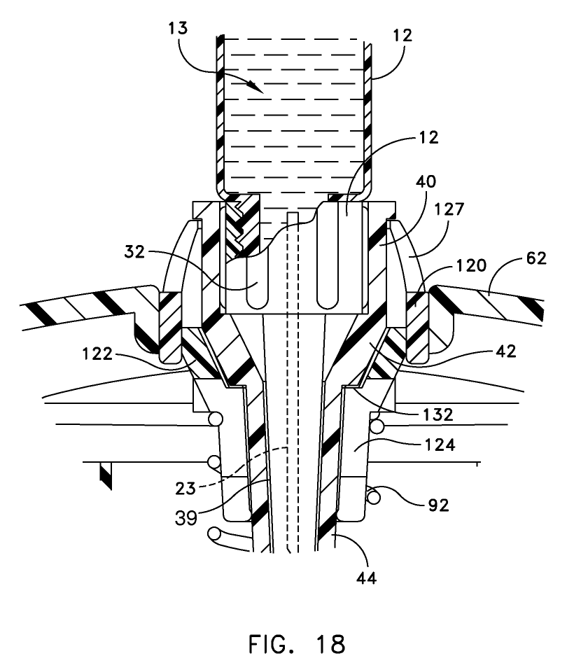

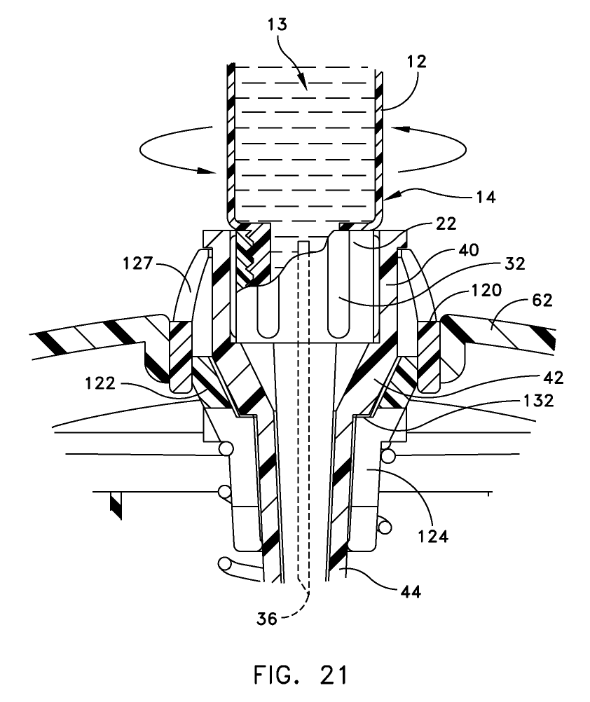

Pod-collet 96 comprises a blind or semi-blind receptacle formed by a cylindrical grip wall 120, an intermediate tapered wall 122, and a elongate tapered wall 124. Cylindrical grip wall 120 comprises a plurality of radially inwardly sloping cantilevered fingers 127 that together define an open end 130 (FIG. 13) that is sized so as to allow for a gripping engagement of a portion of needle-pod 9. In particular, the end edges 133 of cantilevered fingers 127 define an inner diameter of open end 130 that is somewhat smaller than the outer diameter of first cylindrical wall 40 of needle-pod 9 when fingers 127 are in a first position, but define a somewhat larger diameter than first cylindrical wall 40 of needle-pod 9 when in a second position. Intermediate wall 122 is tapered radially inwardly from the bottom end of cylindrical gripper wall 120, and defines an annular shoulder 132 (FIGS. 18 and 21). Elongate tapered wall 124 projects outwardly from the center of shoulder 132, has an open, terminal end 135, and is often somewhat longer and substantially larger in diameter than at least a portion of second cylindrical wall 44 of needle-pod 9. The outer diameter of elongate tapered wall 124 is suitably sized to be received within a distal end section of helical spring 92.

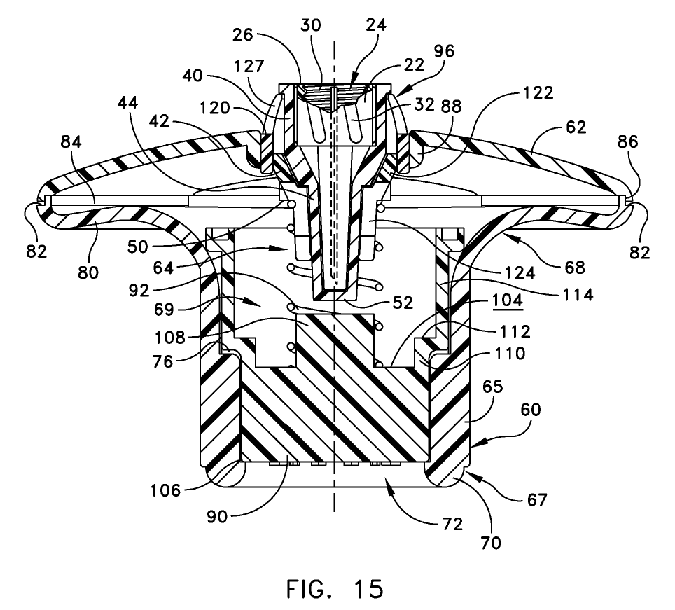

Needle-pod-tool 10 is assembled for use in connection with the present invention in the following manner. Handle 60 is arranged so that the inner surface of hand guard 80 is directed upwardly. A plunger 90 is then positioned in confronting coaxial relation with interior chamber 69 of handle 60. Once in this position, plunger 90 is moved toward handle 60 until peripheral edge 106 of outer surface 102 engages first annular shoulder 74 of wall 65. Once in this position, annular ledge 112 is located in confronting relation to second inner annular shoulder 76 of handle 60. With plunger 90 positioned within handle 60, spring 92 is arranged in confronting coaxial relation to inner surface 104 of plunger 90 so that its proximal end slips over post 108. Spring 92 projects outwardly from around post 108 so that its distal end is substantially coplanar with annular wall 84 of hand guard 80. Also, chordal or inwardly curved edges 81, 85 complementarily line-up so as to form a stabilizer 113 (FIG. 10).

Pod-collet 96 is then arranged in confronting coaxial relation with the central opening of face plate 62. Once in this position, pod-collet 96 is moved toward face plate 62 until cylindrical grip wall 120 snugly engages (press-fits) annular wall 88 that projects outwardly from the rear, concave surface of face plate 62. With pod-collet 96 assembled to face plate 62, the inner surface of hand guard 80 of handle 60 is brought into coaxial confronting relation with the rear, concave surface of face plate 62. Once in this position, face plate 62 is moved toward handle 60, so that elongate tapered wall 124 of pod-collet 96 enters the distal end of spring 92. As this occurs, annular wall 84 engages peripheral recessed groove 86 of face plate 62, so as to enclose interior chamber 69. Face plate 62 is then fixedly attached along the boundary between peripheral recessed groove 86 and annular wall 84 by gluing, ultrasonic welding, or mechanical fastener, as is well-known in the art.

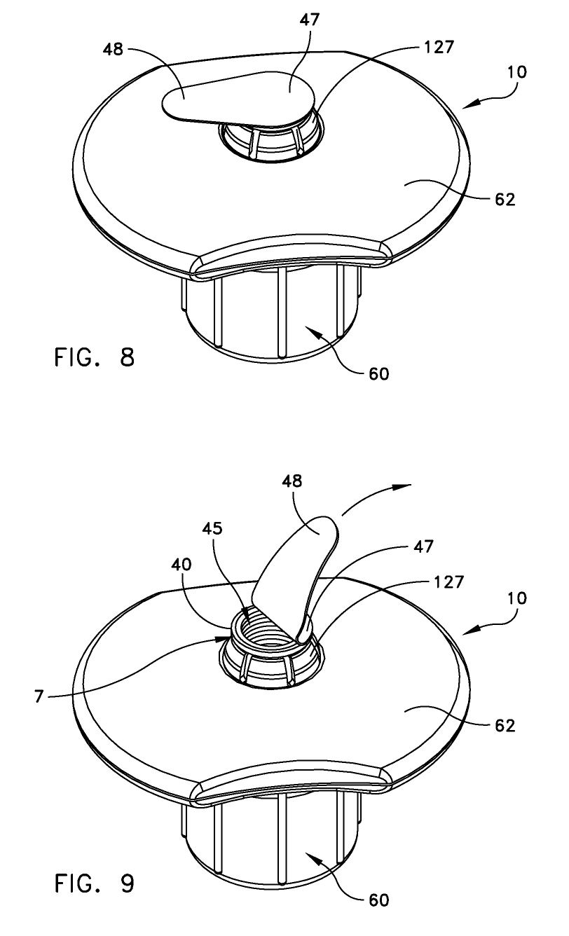

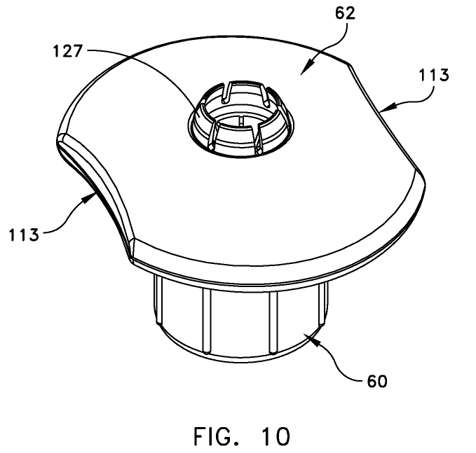

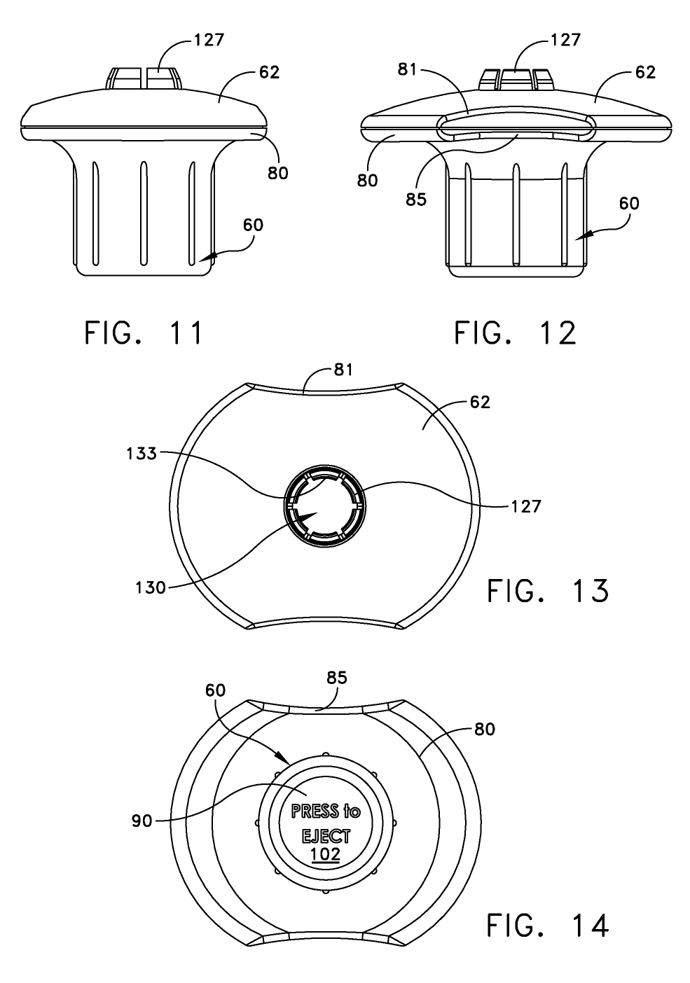

Referring first to FIGS. 5-9, needle dispenser system 3 may be employed to attach a needle assembly 7 to hypodermic syringe 5 in the following manner. A needle-pod 9 is first arranged in confronting coaxial relation with opening 130 of pod-collet 96. Once in this position, needle-pod 9 is moved toward pod-collet 96 so that second cylindrical wall 44 enters opening 130 until cantilevered fingers 127 engage the outer surface of first cylindrical wall 40 of needle-pod 9. It will be understood that due to the difference in respective diameters of opening 130 and first cylindrical wall 40, plurality of cantilevered fingers 127 will be biased radially outwardly by the introduction of needle pod 9 into pod-collet 96 (FIG. 6). Once in this position, needle-pod 9 is firmly gripped within pod-collet 96 by plurality cantilevered fingers 127, with a portion of second cylindrical wall 44 of needle-pod 9 snugly positioned within elongate tapered wall 124 of pod-collet 96 and another portion projecting outwardly from open end 135 (FIGS. 15, 18, 21, 22 and 24). Tab 48 of peelable foil 47 may now be pulled back, thus exposing the top of cylindrical header 22 of needle assembly 7 (FIGS. 8 and 9).

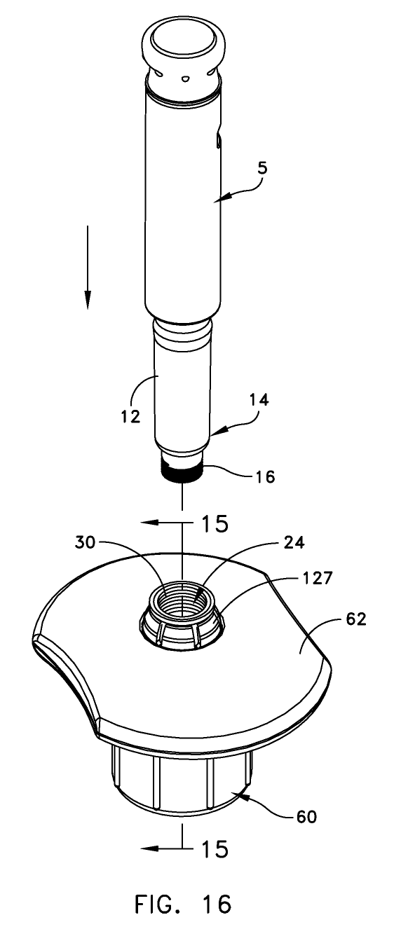

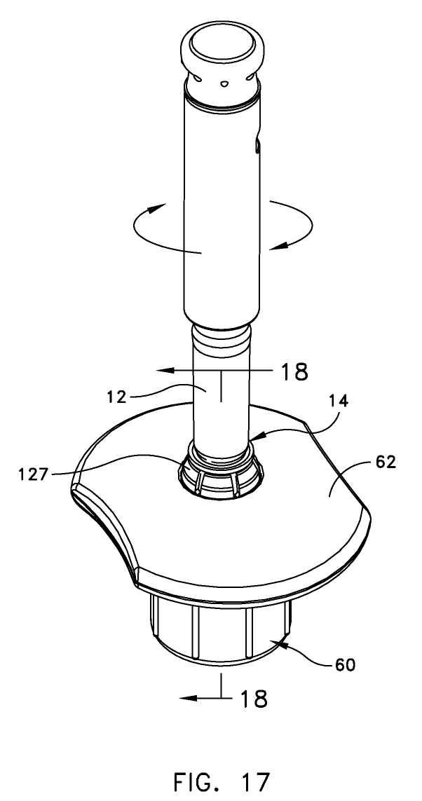

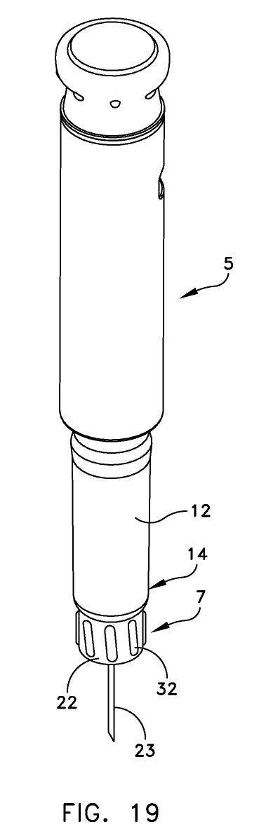

With needle-pod 9 firmly positioned within pod-collet 96, hypodermic syringe 5 may retrieve the needle assembly 7 located within needle-pod 9. More particularly, hypodermic syringe 5 is positioned in confronting coaxial relation with needle-pod 9 (FIG. 16). Hypodermic syringe 5 is then moved toward needle-pod 9, so that threads 16 on distal end 14 engage threads 30 located on the interior surface of first cylindrical wall 40 of needle assembly 7 (FIGS. 16-17). Once in this position, hypodermic syringe 5 is rotated about its longitudinal axis so that threads 16 and 30 engage one another. In this way once threads 16 and 30 are engaged with one another, movement of hypodermic syringe 5 away from face plate 62 of needle-pod-tool 10 provides sufficient force to overcome the press-fit engagement (i.e., a substantially frictional engagement) between plurality of ribs 32 on the outer surface of annular wall 26 and the inner surface of first cylindrical wall 40 of needle-pod 9 (FIGS. 16-20).

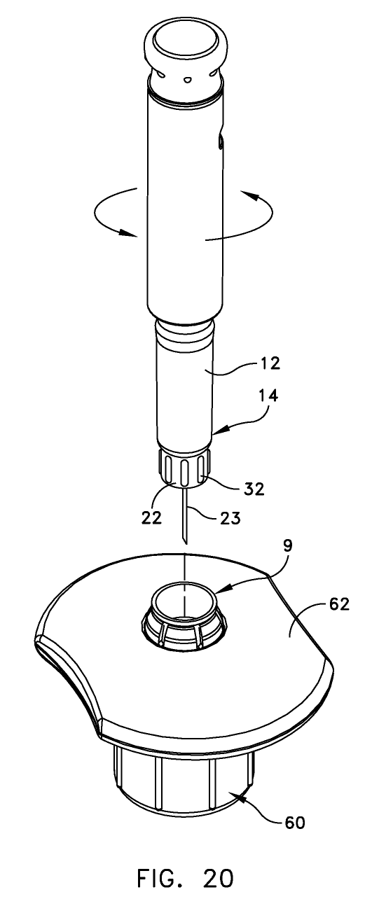

Hypodermic syringe 5 may then be moved to the appropriate location on the patient's body for injection of fluid 13 located within the syringe. Once the injection has been completed, needle assembly 7 may be repositioned within needle-pod 9 and thereby be removed from distal end 14 of hypodermic syringe 5. More particularly, needle 23 along with needle assembly 7 at distal end 14 of hypodermic syringe 5 is first positioned in confronting coaxial relation with needle-pod 9. Once in this position, hypodermic syringe 5 is moved toward needle-pod 9 so that needle assembly 7 reenters needle-pod 9 until plurality of ribs 32 re-engage the interior surface of first cylindrical wall 40. Once in this position, hypodermic syringe 5 is rotated so as to disengage threads 16 from threads 30 (FIGS. 20-22). Once threads 16 are fully disengaged from threads 30, hypodermic syringe 5 may be withdrawn from the vicinity of needle-pod-tool 10, recapped, and repositioned in a pocket or cabinet as desired.

With now-used needle assembly 7 repositioned within needle-pod 9, needle-pod 9 may be removed from needle-pod-tool 10 and discarded. More particularly, needle-pod-tool 10 is first positioned over an appropriate waste receptacle, such that face plate 62 is positioned in spaced confronting relation to the opening of the receptacle. In this position, ejector pad 100 of plunger 90 is visible within the center of opening 72 in handle 60. Once in this position, the patient may press ejector pad 100 with a finger 150 so as to move plunger 90 within interior chamber 69 and move peripheral edge 106 away from first shoulder 74. This, in turn, compressibly biases spring 92 against a portion of pod-collet 96. As plunger 90 moves toward pod-collet 96, post 108 engages the terminal end of elongate tapered wall 124 of needle-pod 9 that defines opening 135. The movement of post 108 against the terminal end of elongate tapered wall 124 is sufficient to overcome the gripping force exerted by plurality of finger cantilevers 127 so as to free and dislodge needle-pod 9 from pod-collet 96 and allow it to fall safely into the receptacle.

It is to be understood that the present invention is by no means limited only to the particular constructions herein disclosed and shown in the drawings, but also comprises any modifications or equivalents within the scope of the claims.

What is claimed is:

1. A needle dispensing system comprising:
a pen-type hypodermic syringe having a threaded end;
a closed pod containing a disposable needle assembly having a threaded portion that is complementary to said threaded end; and
a hand tool including a collet sized so as to releasable grip a portion of said closed pod for opening, and a biased release button the actuation of which causes said opened pod to be released from said collet.

2. A system according to claim 1 wherein said hand tool comprises a handle including a cylindrical wall having a proximal end, a distal end, and which defines an interior chamber.

3. A system according to claim 2 wherein said cylindrical wall has a rounded proximal end edge having a first inner diameter so as to define an opening that is in communication with said interior chamber, a second larger inner diameter adjacent to said first inner diameter so as to define a first annular shoulder, and a third inner diameter adjacent to and larger than said second inner diameter so as to define a second annular shoulder.

4. A system according to claim 3 wherein a distal-most portion of said cylindrical wall flairs radially outwardly so as to form a guard that is sized so as to provide protection so as to avoid needle sticks.

5. A system according to claim 4 wherein a peripheral shoulder is circumferentially defined on a peripheral edge of said guard by an annular wall that projects substantially upwardly from said distal-most, flared portions of said cylindrical wall.

6. A system according to claim 4 wherein said hand tool includes a face plate comprising a convex disk having an outer diameter that is substantially equal to a diameter of said guard, a central opening defined through said face plate by an annular wall that projects outwardly from a rear, concave surface of said face plate, and a peripheral recessed groove that is circumferentially defined in an inner peripheral edge of said face plate that is complementary to said annular wall of said guard.

7. A system according to claim 1 wherein said collet forms a portion of a pod handling assembly including a biased plunger having an ejector pad and a peripheral edge.

8. A system according to claim 7 wherein said biased plunger includes a post that projects outwardly from a surface positioned within an interior chamber of said hand tool.

9. A system according to claim 8 wherein a proximal end of a helical spring is positioned over said post so as to be located within said interior chamber.

10. A system according to claim 4 wherein an annular wall projects upwardly from said peripheral edge of said plunger in radially spaced relation to said post, having an annular ledge that projects radially outwardly from an outer surface, and an annular wall that projects upwardly from a peripheral edge of said annular ledge in radially spaced relation to said post.

11. A system according to claim 1 wherein said collet comprises a semi-blind receptacle formed by a cylindrical grip wall, an intermediate tapered wall that extends from said grip wall, and an elongate tapered wall that projects outwardly from said tapered wall.

12. A system according to claim 11 wherein said grip wall comprises a plurality of radially inwardly sloping cantilevered fingers that together define an open end that is sized so as to allow for a gripping engagement of a portion of said pod.

13. A system according to claim 12 wherein each of said cantilevered fingers includes an end edge that together define an inner diameter of said open end that is somewhat smaller than the outer diameter of a corresponding portion of said pod when said fingers are in a first position, but define a somewhat larger diameter than said corresponding portion of said pod when in a second position.

14. A system according to claim 11 wherein said Intermediate wall is tapered radially inwardly from a bottom end of said gripper wall, and defines an annular shoulder from which said elongate tapered wall projects outwardly.

15. A hand tool for controlling a container holding a disposable needle assembly, comprising:
a housing;
a collet supported by said housing and sized so as to releasably grip a portion of said container for opening, and a biased release button the actuation of which causes said opened pod to be released from said collet.

16. A hand tool according to claim 15 comprising a handle including a cylindrical wall having a proximal end, a distal end, and which defines an interior chamber.

17. A hand tool according to claim 16 wherein said cylindrical wall has a rounded proximal end edge having a first inner diameter so as to define an opening that is in communication with said interior chamber, a second larger inner diameter adjacent to said first inner diameter so as to define a first annular shoulder, and a third inner diameter adjacent to and larger than said second inner diameter so as to define a second annular shoulder.

18. A hand tool according to claim 17 wherein a distal-most portion of said cylindrical wall flairs radially outwardly so as to form a guard.

19. A hand tool according to claim 18 wherein a peripheral shoulder is circumferentially defined on a peripheral edge of said guard by an annular wall that projects substantially upwardly from said distal-most, flared portions of said cylindrical wall.

20. A hand tool according to claim 18 including a face plate comprising a convex disk having an outer diameter that is substantially equal to a diameter of said guard, a central opening defined through said face plate by an annular wall that projects outwardly from a rear, concave surface of said face plate, and a peripheral recessed groove that is circumferentially defined in an inner peripheral edge of said face plate that is complementary to said annular wall of said guard.

21. A hand tool according to claim 15 wherein said collet forms a portion of a pod handling assembly including a biased plunger having an ejector pad and a peripheral edge.

22. A hand tool according to claim 21 wherein said biased plunger includes a post that projects outwardly from a surface positioned within an interior chamber of said hand tool.

23. A hand tool according to claim 22 wherein a proximal end of a helical spring is positioned over said post so as to be located within said interior chamber.

24. A hand tool according to claim 22 wherein an annular wall projects upwardly from said peripheral edge of said plunger in radially spaced relation to said post, having an annular ledge that projects radially outwardly from an outer surface, and an annular wall that projects upwardly from a peripheral edge of said annular ledge in radially spaced relation to said post.

25. A hand tool according to claim 15 wherein said collet comprises a semi-blind receptacle formed by a cylindrical grip wall, an intermediate tapered wall that extends from said grip wall, and an elongate tapered wall that projects outwardly from said tapered wall.

26. A hand tool according to claim 25 wherein said grip wall comprises a plurality of radially inwardly sloping cantilevered fingers that together define an open end that is sized so as to allow for a gripping engagement of a portion of said pod.

27. A hand tool according to claim 26 wherein each of said cantilevered fingers includes an end edge that together define an inner diameter of said open end that is somewhat smaller than the outer diameter of a corresponding portion of said pod when said fingers are in a first position, but define a somewhat larger diameter than said corresponding portion of said pod when in a second position.

28. A hand tool according to claim 21 wherein said Intermediate wall is tapered radially inwardly from a bottom end of said gripper wall, and defines an annular shoulder from which said elongate tapered wall projects outwardly.

29. A needle dispensing system comprising:
a pen-type hypodermic syringe having a threaded end;
a closed pod containing a disposable needle assembly having a threaded portion that is complementary to said threaded end; and
a hand tool having a handle comprising a cylindrical wall that defines an interior chamber and including a rounded proximal end edge having a first inner diameter so as to define an opening that is in communication with said interior chamber, a second larger inner diameter adjacent to said first inner diameter so as to define a first annular shoulder, and a third inner diameter adjacent to and larger than said second inner diameter so as to define a second annular shoulder, wherein a distal-most portion of said cylindrical wall flairs radially outwardly so as to form a guard;
a face plate comprising a convex disk having an outer diameter that is substantially equal to a diameter of said guard, a central opening defined through said face plate by an annular wall that projects outwardly from a rear, concave surface of said face plate, and a peripheral recessed groove that is circumferentially defined in an inner peripheral edge of said face plate that is complementary to said annular wall of said guard; and
a including a collet sized so as to releasable grip a portion of said closed pod for opening, and a biased release button the actuation of which causes said opened pod to be released from said collet.
